(19) United States
(12) Patent Application Publication
(10) Pub. No.: US 2025/0032422 A1
(43) Pub. Date: Jan. 30, 2025

(54) ENCAPSULATED LIQUID PRODUCT AND PROCESS FOR ENCAPSULATING HYDROPHILIC MATERIAL

(71) Applicant: The United States of America as Represented by the Secretary of Agriculture, Washington, DC (US)

(72) Inventors: Hayden Hamby, Fort Collins, CO (US); Abigail Beverly Feuka, Fort Collins, CO (US)

(21) Appl. No.: 18/785,758

(22) Filed: Jul. 26, 2024

Related U.S. Application Data
(60) Provisional application No. 63/528,957, filed on Jul. 26, 2023.

(51) Int. Cl. A61K 9/48; A61K 39/00; A61K 39/04

(52) U.S. Cl. CPC A61K 9/4816; A61K 9/4858; A61K 39/04; A61K 2039/552; A61K 2039/58

(57) ABSTRACT

An encapsulated liquid product and method for encapsulating hydrophilic liquid formulation prepared by crosslinking an alginic acid monomer with calcium lactate for use in administering liquid vaccines, attractants, repellents, toxicants, pesticides, and/or biomarkers to wildlife and livestock. The encapsulated liquid product is edible and biodegradable. Formulations can be produced without expensive machinery. Sublingual or buccal delivery of drugs encapsulating liquid products serves a dual purpose of enhancing mucoadhesion for improved uptake as well as containment prior to ingestion by the target organism. The ability to produce formulations without expensive machinery is currently lacking in the encapsulated liquid materials industry.

### CROSS-REFERENCE TO RELATED APPLICATIONS

This application claims the benefit of U.S. Provisional Patent Application No. 63/528,957, filed on Jul. 26, 2023.

### BACKGROUND

### Field

The disclosure of the present patent application relates to compositions for delivery of hydrophilic liquid formulations. In certain embodiments, the present subject matter relates to an encapsulated liquid product and process for encapsulating hydrophilic material including crosslinking an alginic acid monomer with calcium lactate. The thus produced product can be useful, e.g., in administering liquid vaccines, attractants, repellents, toxicants, pesticides, and biomarkers to wildlife and livestock.

### Description of Related Art

Commercial liquid encapsulation (often referred to as “gelcaps”) are typically manufactured using highly specialized, costly equipment and processes. Accordingly, there is a general need for cost-effective production of encapsulated liquid products and methods for the production thereof. There is increasing interest in the production of encapsulated liquid products. For example, Fortune Business Insights reported a market size for encapsulated liquid products of USD 6.52 billion in 2020.

Encapsulated liquid materials are of particular interest to the food and pharmaceutical industries. For example, in pharmaceutical applications, and, specifically the sublingual/buccal delivery of drugs, encapsulated liquid products serve a dual purpose of enhancing mucoadhesion for improved uptake as well as containment prior to ingestion. The ability to produce such formulations without expensive machinery is currently lacking in the encapsulated liquid materials industry. Moreover, technologies using encapsulated liquid products and methods in the context of wildlife management and animal husbandry is underdeveloped.

Thus, an encapsulated liquid product and process for encapsulating hydrophilic material solving the aforementioned problems is desired.

### SUMMARY

The present subject matter focuses on an encapsulated liquid product. In an embodiment, the present subject matter relates to methods for encapsulating hydrophilic liquid material prepared by crosslinking an alginic acid monomer with calcium lactate for administering to wildlife and livestock. The encapsulated liquid can be prepared by mixing alginic acid monomers with a crosslinking agent, such as calcium lactate. Drops of a composition including at least calcium lactate and the desired encapsulated solution are dispensed into a bath of alginic acid and left to react for a period of time until a gel membrane forms at the surface. The resulting product is then transferred to a water bath to quench the reaction. The resulting product is edible, biodegradable, and mucoadhesive. The resulting product has a gel-like outer membrane encapsulating the hydrophilic liquid formulation. The encapsulated liquid product and methods of producing said encapsulated liquid product solves multiple problems associated with the manufacturing of liquid encapsulates and solid dosage formulations.

In a first aspect, an encapsulated liquid product is provided. The encapsulated liquid product comprises a hydrophilic liquid material prepared by crosslinking an alginic acid monomer with calcium lactate. In various aspects, the encapsulated liquid product is edible, biodegradable, and mucoadhesive. In various aspects, the encapsulated liquid product comprises one or more of a liquid vaccine, attractant, repellent, toxicant, pesticide, and biomarkers. In various aspects, the encapsulated liquid product comprises liquid formulations relevant to wildlife management and animal husbandry. In various aspects, the encapsulated liquid product comprises formulations that can be produced without expensive machinery.

In some aspects, the present method of manufacturing an encapsulated liquid product includes preparing a calcium lactate solution, preparing an alginic acid monomer solution, and adding the calcium lactate solution dropwise to the alginic acid monomer solution. In some embodiments the calcium lactate solution may include calcium lactate and a hydrophilic active agent. The hydrophilic active agent may be one or more of a vaccine, an attractant, a repellent, a toxicant, a pesticide, and a biomarker. In yet further embodiments, the calcium lactate solution may include calcium lactate, a hydrophilic active agent, and a thickener. The thickener may be any thickening agent known in the art, such as, by way of non-limiting example, xanthan gum, psyllium husk, chia seeds, flax seeds, cornstarch, gelatin, egg white, agar, guar gum, baking powder, or the like.

In various aspects, the present subject matter relates to a method of manufacturing an encapsulated liquid product comprising a hydrophilic liquid material prepared by crosslinking an alginic acid monomer with calcium lactate. In various aspects, the method of manufacture can facilitate tailoring the properties of an encapsulated liquid product, including surface properties and size.

In various aspects, a method of manufacturing an encapsulated liquid product comprising a cost-effective means of production. In various aspects, the method of manufacture comprises the use of common laboratory equipment and ingredients that can be readily obtained from unspecialized suppliers or from general retail stores. In various aspects, the method of manufacture eliminates the requirements imposed by the production of encapsulated solid products, which often require specialized extrusion, granulation, drying, and tableting equipment.

In another aspect, a method of delivering an encapsulated liquid product to wildlife and livestock is provided. In various aspects, the encapsulated liquid product facilitates distribution of ingestible liquids to free-ranging wildlife and livestock, including one or more of liquid vaccines, attractants, repellents, toxicants, pesticides, and biomarkers not easily or effectively distributed in solid form.

These and other features of the present subject matter will become readily apparent upon further review of the following specification.

### BRIEF DESCRIPTION OF DRAWINGS

FIG. 1 depicts a flow cytometry analysis of cellular immune responses (in the form of T-cell proliferation) in white tailed deer administered either a negative control (sodium alginate spheres prepared without a vaccine component), a positive control (a BCG vaccine formulated for oral administration), or a test sample (a BCG vaccine encapsulated in a sodium alginate sphere prepared using the present techniques).

FIG. 2 depicts the lack of CD8⁺ T cell proliferative response following consumption of the negative control, positive control, or test sample.

FIG. 3 depicts CD4⁺ T cell proliferative response following consumption of the negative control, positive control, or test sample, when compared to the negative control.

FIG. 4 depicts γδ⁺ T cell proliferative response following consumption of the negative control, positive control, or test sample, when compared to the negative control.

FIG. 5 depicts a graph illustrating the rupture pressure and membrane thickness of sodium alginate spheres as described herein as a function of reaction time.

Similar reference characters denote corresponding features consistently throughout the attached drawings.

### DETAILED DESCRIPTION

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

As used herein, the term “wildlife” refers to any free-ranging, i.e., non-domesticated animal.

As used herein, the term “livestock” refers to any domesticated animal.

### An Encapsulated Liquid Product

The following discussion presents detailed descriptions of the several embodiments of an encapsulated liquid product and processes for encapsulating hydrophilic liquid formulations for use in wildlife and animal husbandry applications. These embodiments are not intended to be limiting, and modifications, variations, combinations, etc., are possible and within the scope of this disclosure.

The present disclosure provides for an encapsulated liquid material and process for encapsulating a hydrophilic liquid product for use, e.g., in wildlife and animal husbandry applications.

The encapsulated liquid product encapsulates a hydrophilic liquid formulation and can be prepared by crosslinking an alginic acid monomer with calcium lactate. The encapsulated liquid product comprises a gel membrane of an alginic acid monomer, calcium lactate and a desired liquid formulation. The desired liquid formulation comprises a liquid or liquid suspension of one or more vaccine, attractant, repellent, toxicant, pesticide, and/or biomarker, and can include combinations thereof. For the avoidance of doubt, a single liquid formulation can include combinations of a single type of agent, i.e., multiple vaccines, combinations of multiple types of agents, i.e., a vaccine and a biomarker, or mixtures thereof. The liquid product is typically encapsulated, i.e., it is typically inside and surround by the gel membrane.

It is another object of the encapsulated liquid product to provide an encapsulated liquid product for use in wildlife and animal husbandry applications. The encapsulated liquid product can enhance mucoadhesion for improved uptake of a liquid formulation by wildlife and livestock, including of a liquid or liquid suspension of one or more vaccines, attractants, repellents, toxicants, pesticides, and/or biomarkers. The encapsulated liquid product can be administered to free-ranging wildlife and/or organisms kept as livestock.

It is another object of the encapsulated liquid product to provide an encapsulated liquid product that is biodegradable.

It is another object of the encapsulated liquid product to provide an encapsulated liquid product that is inexpensive to manufacture. The encapsulated liquid product is typically comprised of cost-effective ingredients that can be readily obtained from unspecialized suppliers or from general retail stores.

It is another object of the encapsulated liquid product to provide an encapsulated liquid product whose properties can be easily tailored. Ease of tailoring facilitates the tuning of encapsulated liquid product properties, including surface properties, size and composition of the desired encapsulated liquid formulation including a liquid or liquid suspension of one or more vaccine, attractant, repellent, toxicant, pesticide, and/or biomarker.

It is another object of the encapsulated liquid product to provide a method of manufacturing an encapsulated liquid product. The method of manufacturing an encapsulated liquid product comprises a gel membrane encapsulating a hydrophilic liquid material prepared by crosslinking an alginic acid monomer with calcium lactate. Drops of calcium lactate with the desired encapsulated formulation are dispensed into a bath of alginic acid monomer and left for a period of time to crosslink with the alginic acid monomer to form a gel membrane, prior to quenching the reaction.

It is another object of the encapsulated liquid product to provide a method of manufacturing an encapsulated liquid product whose properties can be easily modified. The physical properties of the encapsulated liquid product can be easily modified, including by modifying the encapsulated liquid product size, and surface, rupture, and adhesive properties. The composition of the desired encapsulated liquid formulation can also be tailored to specific applications, including by modifying the presence or absence and/or concentration of vaccines, attractants, repellents, toxicants, pesticides, and/or biomarkers in the liquid formulation.

It is another object of the encapsulated liquid product to provide a method of manufacturing an encapsulated liquid product that is cost-effective. The method of manufacture comprises the use of common laboratory equipment and ingredients that can be readily obtained from unspecialized suppliers or from general retail stores. The method of manufacture eliminates the requirements imposed by the production of encapsulated solid products, which often require specialized extrusion, granulation, drying, and tableting equipment.

It is another object of the encapsulated liquid product to provide a method of delivering an encapsulated liquid product to wildlife and livestock. The encapsulated liquid product can facilitate distribution of ingestible liquids to free-ranging wildlife and livestock, including liquid vaccines, attractants, repellents, toxicants, pesticides, and/or biomarkers not easily or effectively distributed in solid form.

In an embodiment, the encapsulated liquid is prepared by mixing alginic acid monomers with a crosslinking agent, such as calcium lactate. Drops of a composition including at least calcium lactate and the desired encapsulated solution are dispensed into a bath of alginic acid and left to react for a period of time until a gel membrane forms at the surface. The resulting product is then transferred to a water bath to quench the reaction. The resulting product is edible, biodegradable, and mucoadhesive. The resulting product has a gel-like outer membrane encapsulating the hydrophilic liquid formulation. The encapsulated liquid product and methods of producing said encapsulated liquid product solves multiple problems associated with the manufacturing of liquid encapsulates and solid dosage formulations.

In a further embodiment, the encapsulated liquid is prepared by mixing alginic acid monomers with a crosslinking agent, such as calcium lactate. In an embodiment, a composition including at least calcium lactate and the desired encapsulated solution is placed in a mold or container sized to hold between about 1 ml and about 12 ml in volume and frozen. The frozen composition can then be dispensed into a bath of alginic acid and left to react for a period of time until a gel membrane forms at the surface. The resulting product can then be transferred to a water bath to quench the reaction. This approach may be particularly useful for delivery of less temperature sensitive desired solutions, including some biologics and toxicants, or where control of the shape of the final product (achievable by selecting the shape of the mold or container used for freezing) is desired. This approach may also be advantageous where a larger volume is desired.

In some aspects, the method of manufacturing an encapsulated liquid product as described herein includes preparing a calcium lactate solution, preparing an alginic acid monomer solution, and adding the calcium lactate solution dropwise to the alginic acid monomer solution. In some embodiments the calcium lactate solution may include calcium lactate and a hydrophilic active agent. The hydrophilic active agent may be one or more of a vaccine, an attractant, a repellent, a toxicant, a pesticide, and a biomarker. In yet further embodiments, the calcium lactate solution may include calcium lactate, a hydrophilic active agent, and a thickener. The thickener may be any thickening agent known in the art, such as by way of non-limiting example xanthan gum, psyllium husk, chia seeds, flax seeds, cornstarch, gelatin, egg white, agar, guar gum, baking powder, or the like.

In an embodiment, either the bath of alginic acid, the composition including at least calcium lactate and the desired encapsulated solution, or both may comprise sugar.

The reaction between the calcium lactate solution and the alginic monomer solution may be allowed to proceed for a period of time prior to quenching the reaction by removing the encapsulated liquid product from the alginic acid monomer solution. In an embodiment, the period of time may be 30 minutes or less, or about 30 minutes or less. In an embodiment, the period of time may be between about 10 minutes and about 30 minutes, or between 10 minutes and 30 minutes. In an embodiment, the period of time may be about 10 minutes, or 10 minutes.

In an embodiment, individual alginate spheres (the encapsulated liquid product) may have a volume between about 1 ml and about 12 ml, or between 1 ml and 12 ml. In an embodiment, the individual alginate spheres may have a volume of about 2 ml, or 2 ml.

Properly made alginate spheres according to any of the present methods may be handled roughly and require sufficient pressure (about 6-16 kpa, or 6-16 kpa) to rupture. The optimal membrane thickness is from about 0.24 mm to about 0.45 mm, or 0.24 mm to 0.45 mm, and has been measured for this work using a caliper under 75 kpa, or under about 75 kpa, for 5 seconds, or about 5 seconds. The final product is best kept in a sealed container under water at 5° C. Storage below 0° C. and in the absence of additional water may result in about 10% moisture loss, or 10% moisture loss, per freeze-thaw cycle and be accompanied by changes to surface texture.

In one embodiment, the encapsulated liquid product may encapsulate a vaccine, including but not limited to the M. bovis bacillus Calmette-Guerin (“BCG”) vaccine. In this embodiment, the encapsulated liquid product may be useful in methods of vaccinating livestock or wildlife against tuberculosis infections.

The encapsulated liquid product and process for encapsulating hydrophilic material may be better understood in view of the following examples.

### Example 1: General Procedure for Making Alginate Spheres

Calcium lactate pentahydrate (Fisher Scientific, USP, ≥98%), xanthan gum (Gum Tech. Coyote Brand, 80 mesh), alginic acid sodium salt (Thermo Scientific, low viscosity, ≤13% w/w loss on drying), and pure cane sugar (C&H Sugar Co., granulated white) were used as received without further purification. Water (18.2 MΩ·cm resistivity and <10 ppb total organic carbon) was obtained via purification with an EMD Millipore Milli-Q system at the National Wildlife Research Center.

A calcium solution/first mixture was prepared as follows. Water (200 g) was added to a 250 mL glass beaker followed by 4.40 g calcium lactate pentahydrate and 10.00 g sugar. This mixture was blended until homogenous using a Polytron PT 10-35 equipped with a Kinematica AG PTA 10TS Aggregate 12 mm head at setting #4 for 30 seconds. Next, 2.00 g xanthan gum was added, and the mixture blended an additional 30 seconds until clumps of gum are no longer present. The resulting solution was opaque and cool to the touch. Prior to use, any air bubbles are removed via centrifugation.

An alginic acid solution/second mixture was prepared as follows. Water (1000 g), 50.00 g sugar, and 10.00 g alginic acid were added to a Waring Commercial Heavy-Duty Blender and blended on the lowest setting in 10 second intervals. Between blending intervals material was scraped from the container walls down into solution and the process repeated until homogeneous. The finished solution was optically clear, pale brown in color, and cool to the touch. Prior to use, any air bubbles were removed by centrifugation or by allowing the solution to rest in an observation dish at room temperature for at least 30 minutes.

The second mixture was poured into a glass observation dish with dimensions of 165 mm diameter and 85 mm height. Aliquots of the first mixture (between about 2 ml and about 2.2 ml in volume) were dispensed into the bath of the second mixture using a 5 mL pipettor equipped with a modified tip such that the opening is 6 mm in diameter. The reaction was allowed to proceed for about 10 minutes before removal and immersion in a water bath. Under these conditions, 10-20 spheres may be prepared at the same time. Ideally, the resulting alginate encapsulates are spherical with no tails or obvious deformities which would compromise structural integrity.

### Example 2: Testing Alginate Spheres for Membrane Thickness and Rupture Pressure

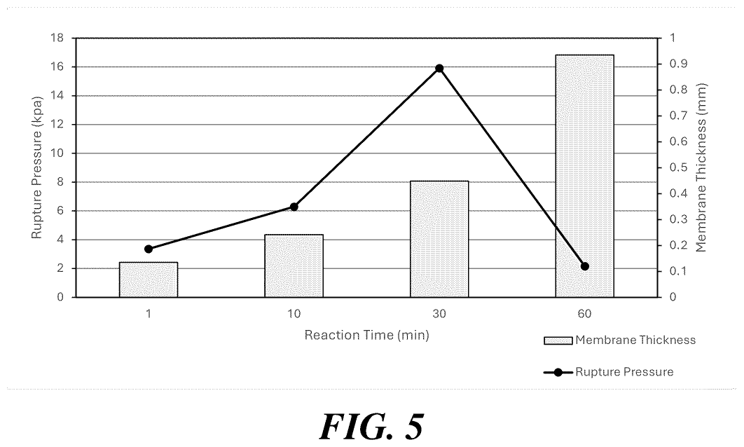

Alginate spheres were prepared according to the general procedure outlined in Example 1, while varying the time that the first mixture was allowed to react with the second mixture before removing the resulting spheres to a water bath to quench the reaction. The procedure was followed while allowing the first and second mixtures to react for about 1 minute, about 10 minutes, about 30 minutes, or about 60 minutes. As shown in FIG. 5, it was surprisingly found that that while membrane thickness and rupture pressure increased with reaction time up to 30 minutes, after 30 minutes membrane thickness continued to increase but rupture pressure was dramatically reduced. It is hypothesized that around the 30-minute mark the alginate spheres become so thick that the membranes become friable, resulting in a dramatic reduction in rupture pressure. Accordingly, it was determined that the optimal time for this step was 30 minutes or less, and it was noted that a reaction time of 10 minutes provided a sufficiently thick and pressure resistant membrane to provide stability to the alginate spheres.

### Example 3: Making Alginate Spheres for BCG Trial

Two separate alginic acid baths were prepared according to the general procedures outlined in Example 1. The first alginic acid bath was prepared using 20.00 g alginic acid, 100.10 g sugar, and 2,000 ml purified water. The second alginic acid bath was prepared using 20.03 g alginic acid, 100.4 g sugar, and 2,000 ml purified water.

A double strength calcium lactate solution was prepared according to the general process outline in Example 1 in two batches. The first batch of double-strength calcium lactate solution was prepared using 26.45 g calcium lactate, 30.04 g sugar, 12.09 g xanthan gum, and 600 ml purified water. The second batch of double-strength calcium lactate solution was prepared using 26.43 g calcium lactate, 30.01 g sugar, 12.01 g xanthan gum, and 600 ml purified water.

A single strength calcium lactate solution was prepared according to the general process outline in Example 1. The single-strength calcium lactate solution was prepared using 7.75 g calcium lactate, 17.50 g sugar, 3.53 g xanthan gum, and 350 ml purified water.

Placebo spheres (negative control, about 2 ml in volume each) were prepared according to the general method outlined in Example 1 using the single strength calcium lactate solution and the alginic acid baths prepared as outlined above.

BCG spheres (test sample, about 2 ml in volume) were prepared by combining equal parts double strength calcium lactate solution with BCG vaccine (250 ml each). The resulting BCG/calcium lactate solution was then used to make sodium alginate spheres according to the general method outlined in Example 1 and using the prepared alginic acid baths.

Additional samples of the oral BCG vaccine used to create the BCG spheres were administered unmodified as a positive control. The oral BCG vaccine used was the M. bovis bacillus Calmette-Guerin (BCG) vaccine first developed for use in humans to prevent or ameliorate M. tuberculosis infections. The BCG vaccine used provided about 10⁸ cfu BCG in 2 ml of media.

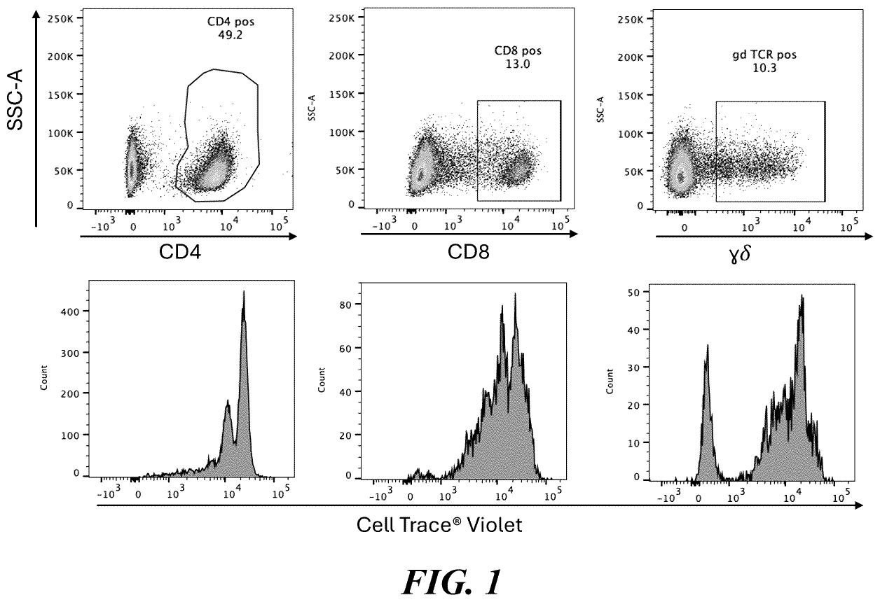

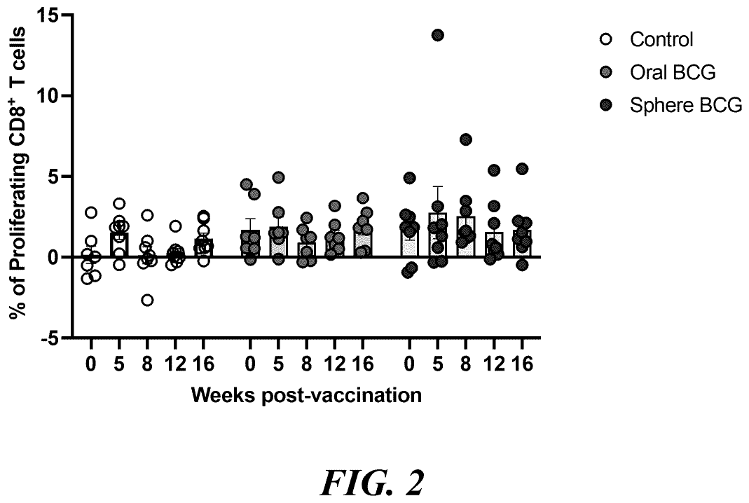

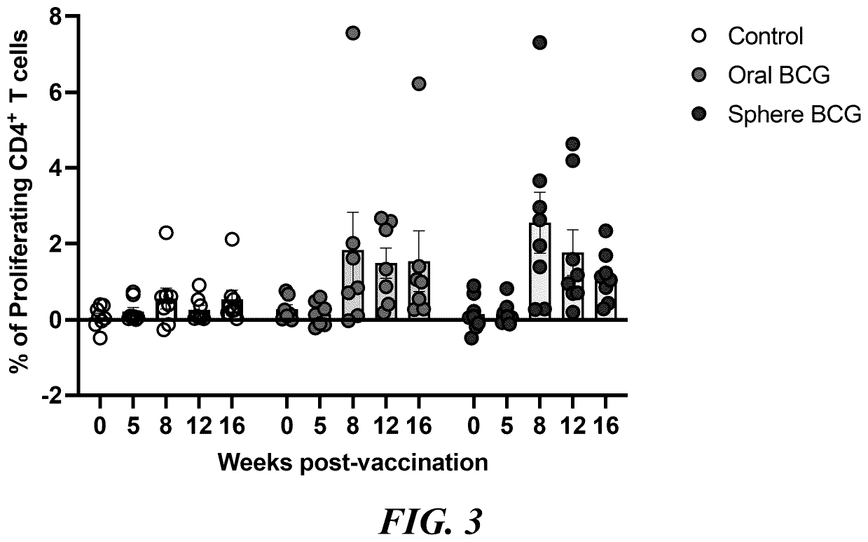

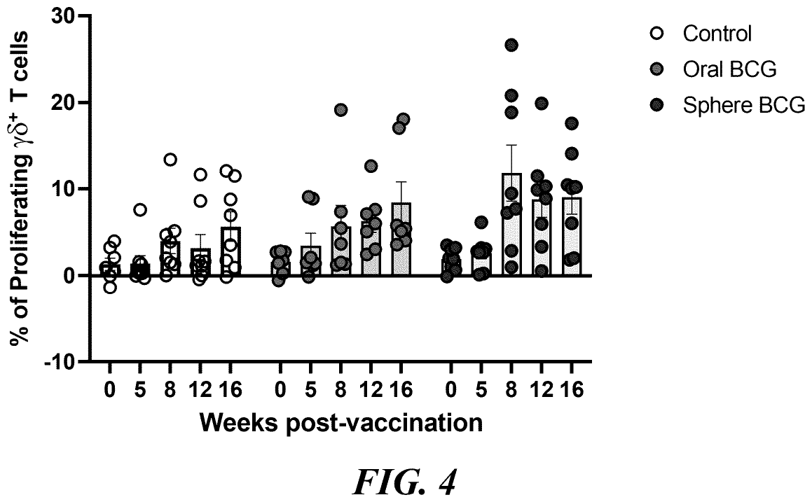

The negative control, positive control, and test samples were each administered orally to white tailed deer and blood samples were collected at 0, 5, 8, 12, and 16 weeks after vaccination. Flow cytometry was used to monitor cellular immune responses, in the form of T-cell proliferation. As shown in FIG. 1, Cell Trace violet was used to identify proliferating cells (dimmer CellTrace readings indicative of cellular division) and non-proliferating cells (brighter CellTrace readings indicative of no cellular division). Staining for CD4, CD8, and the γδ T-cell receptor was also conducted as shown. No measurable CD8⁺ T cell response was observed in any group (See FIG. 2); however, CD4⁺ T cell proliferation was observed in the positive control and test sample as compared to the negative control (see FIG. 3). Further, the CD4⁺ T cell proliferation showed significant variability in individual cellular responses. Finally, γδ T-cells also showed a significant proliferation in response to vaccination with either the positive control or the test sample and showed particularly high individual variability (see FIG. 4). This experiment validated that alginate spheres prepared according to the present teachings are (a) palatable to white-tailed deer and (b) effective for delivery of sufficient numbers of viable bacteria to elicit measurable cellular responses in vaccinated animal.

It is to be understood that the encapsulated liquid product and process for encapsulating hydrophilic material is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An encapsulated liquid product comprising:
a hydrophilic liquid formulation, wherein the hydrophilic liquid formulation comprises:
a liquid or a liquid suspension, wherein the liquid or the liquid suspension comprises one or more of:
a vaccine,
an attractant,
a repellent,
a toxicant,
a pesticide,
a biomarker, and
a combination thereof;
wherein the liquid or the liquid suspension is formulated for administration to wildlife or livestock;
a calcium lactate solution, wherein the calcium lactate solution is mixed with the hydrophilic liquid formulation; and
an alginic acid monomer solution that crosslinks with the calcium lactate solution to form a gel-like membrane that encapsulates the hydrophilic liquid formulation and the calcium lactate solution.

2. The encapsulated liquid product as recited in claim 1, wherein the liquid or the liquid suspension comprises a vaccine.

3. The encapsulated liquid product as recited in claim 2, wherein the vaccine comprises a M. bovis bacillus Calmette-Guerin (“BCG”) vaccine.

4. The encapsulated liquid product as recited in claim 1, comprising between about 1 ml and about 10 ml hydrophilic liquid formulation.

5. The encapsulated liquid product as recited in claim 1, wherein the calcium lactate solution comprises a thickener.

6. The encapsulated liquid product as recited in claim 5, wherein the thickener comprises xanthan gum.

7. The encapsulated liquid product as recited in claim 1, wherein the calcium lactate solution comprises sugar.

8. The encapsulated liquid product as recited in claim 7, wherein the alginic acid monomer solution comprises a sugar.

9. The encapsulated liquid product as recited in claim 1, wherein the alginic acid monomer solution comprises a sugar.

10. The encapsulated liquid product as recited in claim 1, wherein the gel like membrane has a thickness between about 0.24 mm and about 0.45 mm.

11. A method of encapsulating a liquid product comprising:
preparing a first solution comprising calcium lactate;
preparing a bath comprising a second solution comprising an alginic acid monomer;
preparing a hydrophilic liquid formulation, wherein the hydrophilic liquid formulation comprises:
a liquid or a liquid suspension, wherein the liquid or the liquid suspension comprises one or more of:
a vaccine,
an attractant,
a repellent,
a toxicant,
a pesticide,
a biomarker, and
a combination thereof;
wherein the liquid or the liquid suspension is formulated for administration to wildlife or livestock;
combining the first solution and the hydrophilic liquid formulation to form a combined solution;
adding the combined solution to the bath comprising the second solution using dropwise addition;
allowing cross-linking of the calcium lactate in the combined solution with the alginic acid monomer in the second solution to proceed for a period of time to form a gel-like membrane encapsulating the first solution and the hydrophilic liquid formulation; and
quenching the cross-linking reaction by removing the gel-like membrane from the second solution.

12. The method as recited in claim 11, wherein the period of time is 30 minutes or less.

13. The method as recited in claim 12, wherein the period of time is about 10 minutes.

14. The method as recited in claim 11, wherein the first solution comprises a thickener.

15. The method as recited in claim 14, wherein the thickener comprises xanthan gum.

16. The method as recited in claim 11, wherein the first solution comprises a sugar.

17. The method as recited in claim 16, wherein the second solution comprises a sugar.

18. The method as recited in claim 11, wherein the second solution comprises a sugar.

19. A method of administering a hydrophilic liquid formulation to wildlife or livestock comprising:
delivering an encapsulated liquid product to a target organism,
the encapsulated liquid product comprising an alginic acid monomer membrane that encapsulates a solution of calcium lactate and the hydrophilic liquid formulation, wherein the hydrophilic liquid formulation comprises:
a liquid or a liquid suspension, wherein the liquid or the liquid suspension comprises one or more of:
a vaccine,
an attractant,
a repellent,
a toxicant,
a pesticide,
a biomarker, and
a combination thereof; and
wherein the target organism comprises either wildlife or livestock.

20. The method as recited in claim 19, wherein the encapsulated liquid product comprises a thickener and sugar.
